(19) United States
(12) Patent Application Publication
(10) Pub. No.: US 2016/0310404 A1
(43) Pub. Date: Oct. 27, 2016

(54) WASH-OUT HAIR TREATMENT COMPOSITION

(71) Applicant: KAO USA, Inc., Cincinnati, OH (US)

(72) Inventors: Adam SCHROTT, Cincinnati, OH (US); Christopher Holmes, Cincinnati, OH (US)

(21) Appl. No.: 15/132,320

(22) Filed: Apr. 19, 2016

Related U.S. Application Data
(60) Provisional application No. 62/150,489, filed on Apr. 21, 2015.

(51) Int. Cl. A61K 8/898; A61K 8/73; A61K 8/81; A61Q 5/12; A61K 8/34; A61K 8/365; A61K 8/362; A61K 8/891

(52) U.S. Cl. CPC A61K 8/898; A61K 8/362; A61K 8/73; A61K 8/891; A61Q 5/12; A61K 8/345; A61K 8/365; A61K 8/731; A61K 8/8147; A61K 2800/594; A61K 2800/80; A61K 2800/592; A61K 2800/30

(57) ABSTRACT

Wash-out hair treatment compositions are disclosed. The compositions comprise a cosmetically-acceptable suspending agent (such as xanthan gum), a cosmetically-acceptable organic acid (such as succinic acid or gluconic acid), silicone having amino, aminopropyl and/or quaternary ammonium functional groups (such as bis-cetearyl amodimethicone), and a hair care carrier; the pH of the compositions being within about 0.75 pH units of the pKa1 of the selected organic acid or mixtures of organic acids. The method of using the defined compositions to provide hair care benefits which can last through several shampoos is also disclosed.

This application is based upon and claims priority from U.S. Provisional Patent Application 62/150,489, Schrott and Holmes, filed Apr. 21, 2015, incorporated herein by reference.

### BACKGROUND

The present invention relates to compositions which are applied to the hair and subsequently rinsed out, thereby providing long-lasting hair conditioning benefits (for example, benefits lasting through 3 to 5 shampoos).

Consumers want their hair to have excellent tactile properties (smooth and soft), be tame and behaved (free from frizz), and be responsive to manipulation during styling. The present invention is unique in that it not only delivers these benefits, but delivers them from a wash-out product such that the benefits last through multiple (3 to 5) shampoos.

Existing products which are said to provide the benefits of the present invention generally are leave-on products which attempt to provide the desired benefits through the application of:
high viscosity dimethicone/dimethiconols (>100,000 cst)—however, those materials can leave hair feeling greasy and weigh the hair down; and/or
conventional hair styling polymers, which can make the hair feel stiff (a negative consumer attribute).

In addition, these existing products typically provide transient benefits, lasting only through the next shampoo.

### BRIEF SUMMARY

The present invention accomplishes this highly desirable long-lasting result through the combination of a defined organic acid, a defined suspending agent, and a defined silicone material, at a defined pH. The following components are used to prepare the compositions of the present invention:

(a) from about 0.1% to about 5.0% (by weight), for example from about 0.5% to about 2.0% (by weight), of a cosmetically-acceptable suspending agent, such as those selected from xanthan gum, hydroxypropyl methyl cellulose, hydroxyethyl cellulose, guar gum, hydroxypropyl guar gum, starch, starch derivatives, and mixtures thereof;

(b) from about 0.1% to about 7.5% (by weight), such as from about 0.5% to about 5.0% (by weight), of a cosmetically-acceptable organic acid, such as those selected from succinic acid, malic acid, lactic acid, gluconic acid, citric acid, and mixtures thereof;

(c) from about 1% to about 10% (by weight) of a silicone having one or more functional groups selected from amino-, aminopropyl-, or quaternary ammonium (which may or may not contain other functional groups, such as methoxy, hydroxyl, ethoxy, cetearyl, methyl, ethyl or phenyl); and

(d) a hair care carrier;

the final pH of the composition must be such that it is within about 0.75 pH units of the pKa1 of the selected acid or mixture of acids. The composition pH may be adjusted, if need be, using sodium hydroxide, monoethanolamine, or another cosmetically-acceptable base.

The method of using those compositions to provide long-lasting hair conditioning benefits is also claimed.

### DETAILED DESCRIPTION

The wash-out hair conditioning compositions herein include at least the following components: (a) one or more cosmetically-acceptable suspending agents; (b) one or more cosmetically-acceptable organic acids; (c) one or more silicones having one or more functional groups selected from amino, aminopropyl and quaternary ammonium groups; and (d) a hair care carrier.

As used herein, “cosmetically-acceptable” means a material which is safe for application to a person's head, hair, skin and face, and which is compatible with the other components of the defined hair conditioning composition.

As used herein, all percentages and ratios are “by weight” unless otherwise specified. Further, all patents and publications referenced in this application are incorporated by reference herein in their entirety.

The present invention includes a silicone material which includes functional groups selected from amino groups, aminopropyl groups, quaternary ammonium groups, and combinations of those groups (i.e., an aminosilicone). The silicone materials are generally present at from about 1% to about 10%, for example, from about 2% to about 7.5% (by weight) of the defined composition.

As used herein, “aminosilicone” means any polysiloxane having at least one primary, secondary or tertiary amine function or a quaternary ammonium group. Examples of useful aminosilicones are described in PCT Published Patent Application WO 2015/021516, Biato (L'Oreal), published Feb. 19, 2015, incorporated herein by reference.

Preferably, the aminosilicones useful in the present invention are selected from:

the compounds corresponding to the following formula (I):

(R¹)ₐ(T)₃₋ₐ—Si[OSi(T)₂]ₙ—[OSi(T)_{b}(R¹)_{2-b}]ₘ—OSi(T)₃₋ₐ—(R¹)ₐ (I)

wherein:

T is a hydrogen atom, or a phenyl, hydroxyl (—OH), or C₁-C₈ alkyl radical, such as methyl or C₁-C₈ alkoxy, preferably methoxy; a denotes the number 0 or an integer from 1 to 3, and preferably 0; b denotes 0 or 1, and in particular 1;

m and n are numbers such that their sum (n+m) can vary from 1 to 2000, and in particular from 50 to 150, with n denoting a number from 0 to 1999 and notably from 49 to 149, and m denoting a number from 1 to 2000, and notably from 1 to 10;

R¹ is a monovalent radical of formula —C_{q}H_{2q}L, in which q is a number from 2 to 8 and L is an amino group, optionally quaternized, selected from the groups:

—N(R²)—CH₂—CH₂—N(R²)₂;

—N(R²)₂; —N⁺(R²)₃Q⁻;

—N⁺(R²) (H)₂ Q⁻;

—N⁺(R²)₂HQ⁻; and

—N(R²)—CH₂—CH₂—N⁺(R²)(H)₂Q⁻;

in which R² denotes a hydrogen atom, a phenyl, a benzyl, or a saturated monovalent hydrocarbon radical, for example a C₁-C₂₀ alkyl radical, and Q⁻ represents a halide ion such as, for example, fluoride, chloride, bromide or iodide.

In particular, the aminosilicones corresponding to the definition of formula (I) are selected from the compounds having the following formula (Ia):

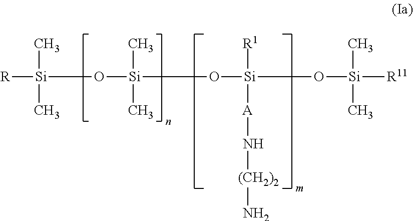

in which R, R¹, R¹¹, which may be identical or different, denote a C₁-C₄ alkyl radical, preferably CH₃; a C₁-C₄ alkoxy radical, preferably methoxy; or OH; A represents a linear or branched C₃-C₈, preferably C₃-C₆, alkylene radical; m and n are integers that depend on and define the molecular weight of the molecule, and whose sum is preferably between 1 and 2000.

According to a first embodiment, R, R¹, R¹¹, which may be identical or different, represent a C₁-C₄ alkyl radical or hydroxyl radical, A represents a C₃ alkylene radical, and m and n are such that the weight-average molecular weight of the compound is between about 5000 and about 500,000. Compounds of this type are called “amodimethicone” in the INCI Dictionary.

According to a second embodiment, R, R¹, R¹¹, which may be identical or different, represent a C₁-C₄ alkoxy radical or hydroxyl radical, at least one of the radicals R or R¹¹ is an alkoxy radical and A represents a C₃-C₄ alkylene radical. The hydroxyl/alkoxy molar ratio is preferably between about 0.2/1 and about 0.4/1 and advantageously is equal to about 0.3/1. Moreover, m and n are such that the weight-average molecular weight of the compound is between 2000 and 10⁶. More particularly, n is between 0 and 999, and m is between 1 and 1000, the sum of n and m being between 1 and 1000.

This category of compounds includes, among others, the product Belsil ADM® 652, marketed by Wacker.

According to a third embodiment, R, R¹¹, which are different, represent a C₁-C₄ alkoxy radical or hydroxyl radical, at least one of the radicals R, R¹¹ is an alkoxy radical, R¹ represents a methyl radical and A represents a C₃-C₄ alkylene radical. The hydroxyl/alkoxy molar ratio is preferably between about 1/0.8 and about 1/1.1, and advantageously is equal to about 1/0.95. Moreover, m and n are such that the weight-average molecular weight of the compound is between 2000 and 200000. More particularly, n is between 0 and 999, and m is between 1 and 1000, the sum of n and m being between 1 and 1000.

This category includes, among others, the product FluidWR® 1300, marketed by Wacker.

According to a fourth embodiment, R, R¹¹ represent a hydroxyl radical, R¹ represents a methyl radical and A is a C₃-C₈, preferably C₃-C₄, alkylene radical. Moreover, m and n are such that the weight-average molecular weight of the compound is between 2000 and 10⁶. More particularly, n is between 0 and 1999 and m is between 1 and 2000, the sum of n and m being between 1 and 2000.

A product of this type, for example, is marketed under the name DC28299 by Dow Corning.

The molecular weight of these silicones is determined by gel permeation chromatography (room temperature, polystyrene standard; columns μ styragem; eluent THF; flow of 1 mm/m; inject 200 μl of a 0.5 wt. % solution of silicone in THF and use detection by refractometry and with a UV-meter).

An aminosilicone corresponding to the definition of formula (I) can also be a polymer called “trimethylsilylamodimethicone” in the INCI Dictionary (16th edition, 2016), and having the following formula (Ib):

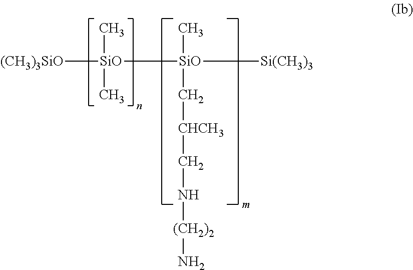

wherein n and m have the meanings given above according to formula (I) or (Ia).

Such compounds are described, for example, in EPO Published Patent Application EP0095238, incorporated by reference herein; a compound of formula (Ib) is, for example, sold under the name Q2-8220 by the company OSI.

Aminosilicones may have the following formula (II):

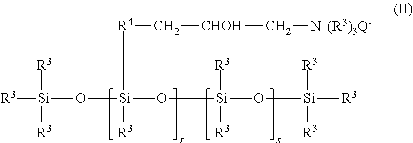

wherein:

R³ represents a monovalent C₁-C₁₈ hydrocarbon radical, and preferably a C₁-C₁₈ alkyl radical or C₂-C₁₈ alkenyl radical, for example methyl;

R⁴ represents a divalent hydrocarbon radical, notably a C₁-C₁₈ alkylene radical or a divalent C₁-C₁₈, for example C₁-C₁₈ alkyleneoxy radical;

Q⁻ is a halide ion, for example chloride;

r represents an average random value from 2 to 20, and in particular from 2 to 8;

s represents an average random value from 20 to 200, and in particular from 20 to 50.

Such compounds are described more particularly in U.S. Pat. No. 4,185,087, Morlino, issued Jan. 22, 1980, incorporated by reference herein.

Quaternary ammonium silicones may have the following formula (III)

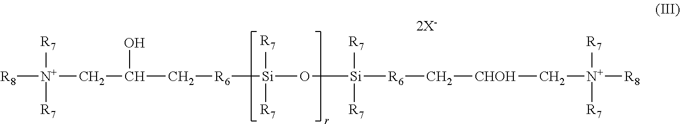

wherein:

R₇, which may be identical or different, represents a monovalent hydrocarbon radical having from 1 to 18 carbon atoms, and in particular C₁-C₁₈ alkyl radical, a C₂-C₁₈ alkenyl radical, or a ring comprising 5 or 6 carbon atoms, for example methyl;

R₆ represents a divalent hydrocarbon radical, notably a C₁-C₁₈ alkylene radical or a divalent C₁-C₁₈, for example C₁-C₈, alkyleneoxy radical joined to the Si by an SiC bond;

R₈, which may be identical or different, represents a hydrogen atom, a monovalent hydrocarbon radical having from 1 to 18 carbon atoms, and in particular a C₁-C₁₈ alkyl radical, a C₂-C₁₈ alkenyl radical, or a radical —R₆—NHCOR₇;

X⁻ is an anion such as a halide ion, for example chloride, or a salt of an organic acid (acetate, etc.);

r represents an average random value from 2 to 200, and in particular from 5 to 100.

These silicones are, for example, described in EPO Published Patent Application EP-A-0530974, Birtwistle (Unilever), published Mar. 10, 1993, incorporated herein by reference.

As an example of a compound of formula (III), mention may be made of the product referred to in the INCI Dictionary (2016 Edition) under the name Quaternium 80, such as that offered by the company EVONIK GOLDSCHMIDT, under the names ABIL QUAT 3272 or 3474.

Aminosilicones may have the following formula (IV):

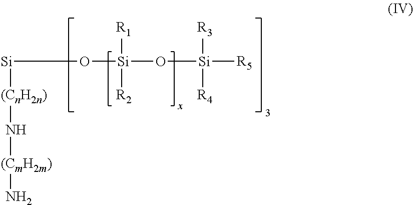

wherein:

R₁, R₂, R₃ and R₄, which may be identical or different, denote a C₁-C₄ alkyl radical or a phenyl group;

R₅ denotes a C₁-C₄ alkyl radical or a hydroxyl group;

n is an integer in the range from 1 to 5;

m is an integer in the range from 1 to 5;

and in which x is selected in such a way that the amine index is between 0.01 and 1 meq/g.

Aminosilicones used in the present invention are preferably aminosilicones of formula (I), (Ia) or (Ib).

Examples of aminosilicones useful in the present invention include amodimethicone, bis-cetearyl amodimethicone, bis-hydroxy/methoxy amodimethicone, aminopropyl phenyl trimethicone, quaternium-80, silicone quaternium-18, silicone quaternium-16/glycidoxy dimethicone crosspolymer, and mixtures thereof.

The second component included in the present invention is one or more cosmetically-acceptable organic acids. Those acids are included in the present compositions at from about 0.1% to about 7.5% (by weight), such as from about 0.5% to about 5.0% (by weight), of the compositions. Such acids are well-known in the art and are generally selected from a-carboxylic acids, multivalent carboxylic acids (e.g., 1,2-dicarboxylic acids and/or 1,3-dicarboxylic acids), and mixtures thereof. Examples include succinic acid, malic acid, lactic acid, gluconic acid, citric acid, and mixtures thereof.

The pH of the defined compositions must be such that it is within about 0.75 pH units of the pKa1 of the selected organic acid or mixture of organic acids. For example, when the organic acid is succinate acid, the composition pH is from about 3.4 to about 4.9. pKa is a term well-known in the chemical arts. When an acid can lose more than one proton, the pKa1 is the pH at which the first proton is lost. The composition pH may be adjusted, if need be, by the addition of sodium hydroxide, monoethanolamine or another cosmetically-acceptable base.

The third component included in the present invention is from about 0.1% to about 5.0% (by weight), such as from about 0.5% to about 2.0% (by weight), of a cosmetically-acceptable suspending agent. Suspending agents for silicone materials are well-known in the hair shampoo/conditioner art and, for example, are described in U.S. Pat. No. 5,948,739, Inman, issued Sep. 7, 1999; U.S. Pat. No. 4,741,855, Grote et al, issued May 3, 1988; U.S. Pat. No. 4,788,006, Bolich et al, issued Nov. 29, 1988; U.S. Pat. No. 4,704,272, Oh et al, issued Nov. 3, 1987; and U.S. Pat. No. 2,798,053, Brown, issued Jul. 2, 1957; all of which are incorporated herein by reference. Examples of useful suspending agents include xanthan gum, hydroxypropyl methyl cellulose, hydroxyethyl cellulose, guar gum, hydroxypropyl guar gum, starch, starch derivatives, and mixtures thereof. As can be seen from these examples, the suspending agents can be nonionic, anionic or cationic in nature; they are chosen for their compatibility with the remainder of the particular hair conditioner formulation. In preferred compositions the weight ratio of the suspending agent to the organic acid is from about 1:2 to about 5:1.

The compositions of the present invention can also optionally include a sugar alcohol, such as glycerol, erythritol, mannitol and/or sorbitol, for example at from about 5% to about 15% of the compositions.

The balance of the compositions of the present invention is made up of a hair care carrier which is compatible with the composition ingredients, as well as with the hair and skin (as well as the eyes) of the user. Examples of such carriers include water, ethanol, glycol, and water/ethanol mixtures. The carrier can, for example, comprise 90% or more of the finished composition.

The compositions of the present invention generally are substantially free (i.e., less than 0.5%, preferably less than 0.05%, more preferably less than 0.001%) of from conventional hair styling polymers, such as those described in U.S. Pat. No. 5,104,642, Wells et al, issued Apr. 14, 1992, incorporated herein by reference. Examples of such polymers include styrene/acrylates copolymer, polyurethanes, octylacrylamide/acrylates/butylaminoethyl methacrylate copolymer, acrylates/t-butylacrylamide copolymer, and acrylate copolymers.

Other conventional hair care ingredients, such as quaternary alkyl ammonium compounds, esters, oils, emollients, humectants, dimethicones, dyes, perfumes and preservatives, may be included in the present invention at their art-established usage levels, to obtain their art-established benefits.

The compositions of the present invention are used by applying a hair conditioning effective amount (such as about 10 to about 240 grams) of the composition to dry or damp hair, letting it sit on the hair for about 5-20 minutes (for example, from about 10 to about 15 minutes), and then shampooing the composition off. Not only does the composition provide excellent tactile, frizz and styling properties, but those properties last through multiple shampoos.

### EXAMPLES

Examples of the compositions of the present invention are set forth in the following table. These compositions are intended to be exemplary only, and are not intended to be limiting of the present invention.

These compositions are made as follows:

| | 1 | 2 | 3 | 4 | 5 | 6 |
|---|---|---|---|---|---|---|
| Deionized Water | Balance | | | | | |
| Ethanol | 15.0 | 5.0 | 10.0 | | 5.0 | |
| Succinic Acid | 1.0 | | | | 0.5 | |
| Lactic Acid | | 1.0 | | | | 0.5 |
| Malic Acid | | | 2.0 | | | 0.5 |
| Citric Acid | | | | 1.0 | 0.5 | |
| Xanthan Gum | 1.0 | | | | | |
| Hydroxypropyl Methyl Cellulose | | 0.8 | | | | |
| Hydroxyethyl Cellulose | | | 1.5 | | | |
| Polyquaternium-52 | | | | 1.5 | | 0.5 |
| Polyquaternium-37 | | | | | 0.5 | 0.3 |
| Dimethicone | 5.0 | | | 2.5 | | |
| Bis-Hydroxy/Methoxy Amodimethicone | 1.0 | 1.0 | | | 3.5 | |
| Amodimethicone | | 1.0 | | | | 2.5 |
| Bis-Cetearyl Amodimethicone | | | 3.0 | | | |
| Aminopropyl Phenyl Trimethicone | | | | 5.0 | | |

| | |
|---|---|
| Fragrance | QS |
| Preservative | QS |
| pH | Adjust to pKa1 +/− 0.75 |

When an effective amount of those compositions is applied and worked into dry or damp hair, allowed to sit on the hair for about 5-12 minutes, and then shampooed out of the hair, the compositions provide the hair with excellent tactile, frizz and styling properties which last through several shampoos.

What is claimed is:

1. A hair care composition comprising:
(a) from about 0.1% to about 5.0% of a cosmetically-acceptable suspending agent selected from xanthan gum, hydroxypropyl methyl cellulose, hydroxyethyl cellulose, guar gum, hydroxypropyl guar gum, starch, starch derivative, and mixtures thereof;
(b) from about 0.1% to about 7.5% of an organic acid selected from succinic acid, malic acid, lactic acid, gluconic acid, citric acid, and mixtures thereof;
(c) from about 1% to about 10% of one or more silicones with one or more functional groups selected from amino, aminopropyl, quaternary nitrogen groups; and
(d) a suitable hair care carrier;
wherein the pH of the composition is within about 0.75 pH units of the pKa1 of the selected organic acid or mixture of organic acids.

2. The composition of claim 1 wherein said suspending agent is present from about 0.5% to about 2.0% by weight.

3. The composition of claim 1 wherein the weight ratio of said suspending agent to said organic acid is from about 1:2 to about 5:1.

4. The composition of claim 1 wherein said organic acid is selected from succinic acid, lactic acid, and mixtures thereof.

5. The composition of claim 4, wherein said organic acid is succinic acid.

6. The composition of claim 5 wherein pH of composition is from about 3.4 to about 4.9.

7. The composition of claim 1 wherein said organic acid is present from about 0.5% to about 5.0% by weight.

8. The composition of claim 1 wherein said silicone is selected from the group consisting of amodimethicone, bis-cetearyl amodimethicone, bis-hydroxy/methoxy amodimethicone, quaternium-80, silicone quaternium-18, silicone quaternium-16/glycidoxy dimethicone crosspolymer, and mixtures thereof.

9. The composition of claim 7 wherein said silicone is bis-hydroxy/methoxy amodimethicone.

10. The composition of claim 7 wherein said silicone is bis-cetearyl amodimethicone.

11. The composition of claim 1 further comprising a sugar alcohol.

12. The composition of claim 11 wherein the sugar alcohol is present from about 5% to about 15%.

13. The method of providing hair conditioner benefits to hair comprising the steps of:
(a) applying a hair conditioner effective amount of the composition of claim 1 to dry or damp hair
(b) letting the composition remain on the hair for about 5 to about 20 minutes; and
(c) rinsing the hair.

14. The method of claim 11 wherein compositions is applied to dry hair.

15. The method of claim 11 further comprising shampooing the hair after said rinse.
